SEQUENCE LISTING (10 sequences)

SEQ ID NO: 1 - RNA, 21 nt, Artificial Sequence
  hsa-miR-106b-5p (mature sequence)
uaaagugcugacagugcagau

SEQ ID NO: 2 - RNA, 22 nt, Artificial Sequence
  hsa-miR-106b-3p
ccgcacuguggguacuugcugc

SEQ ID NO: 3 - RNA, 22 nt, Artificial Sequence
  hsa-miR-93-5p (mature sequence)
aaagugcuguucgugcagguag

SEQ ID NO: 4 - RNA, 23 nt, Artificial Sequence
  hsa-miR-93-5p (pre-miR-93 sequence)
caaagugcuguucgugcagguag

SEQ ID NO: 5 - RNA, 22 nt, Artificial Sequence
  hsa-miR-93-3p
acugcugagcuagcacuucccg

SEQ ID NO: 6 - RNA, 21 nt, Artificial Sequence
  reverse complement of hsa-miR-106b-5p
aucugcacugucagcacuuua

SEQ ID NO: 7 - RNA, 22 nt, Artificial Sequence
  Reverse complement of hsa-miR 106b-3p
gcagcaaguacccacagugcgg

SEQ ID NO: 8 - DNA, 4385 bp, Dependoparvovirus
  misc_feature (1)..(4385): AAV9 genome
cagagagggagtggccaactccatcactaggggtaatcgcgaagcgcctcccacgctgccgcgtcagcgctgacgtagattacgtcataggggagtggtcctgtattagctgtcacgtgagtgcttttgcgacattttgcgacaccacatggccatttgaggtatatatggccgagtgagcgagcaggatctccattttgaccgcgaaatttgaacgagcagcagccatgccgggcttctacgagattgtgatcaaggtgccgagcgacctggacgagcacctgccgggcatttctgactcttttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggagaagctggtccagaccatctaccgcgggatcgagccgaccctgcccaactggttcgcggtgaccaagacgcgtaatggcgccggcggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtaggcccttcacttccggtggacattacgcagaaccgcatctaccgcatcctgcagctcaacggctacgaccctgcctacgccggctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccactcccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgcttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacggcaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccaaaggtgtttactctgagcctcgccccattggtactcgttacctcacccgtaatttgtaattgcctgttaatcaataaaccggttaattcgtttcagttgaactttggtctctgcg

SEQ ID NO: 9 - DNA, 4393 bp, Dependoparvovirus
  misc_feaeture (1)..(4393): AAV8 genome
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 10 - RNA, 22 nt, Artificial Sequence
  Scrambled sequence
aaggcaagcugacccugaaguu

(19) United States
(12) Patent Application Publication
(10) Pub. No.: US 2024/0294909 A1
(43) Pub. Date: Sep. 5, 2024

(54) AGENTS, COMPOSITIONS, AND METHODS FOR THE TREATMENT OF HYPOXIA AND ISCHEMIA-RELATED DISORDERS

(71) Applicant: MERAND PHARMACEUTICALS, INC., Charlottesville, VA (US)

(72) Inventor: Brian H. Annex, Augusta, GA (US)

(21) Appl. No.: 18/276,899

(22) PCT Filed: Feb. 11, 2022
(86) PCT No.: PCT/US22/16228

§ 371 Date: Aug. 11, 2023

Related U.S. Application Data
(60) Provisional application No. 63/148,940, filed on Feb. 12, 2021.

(51) Int. Cl. C12N 15/113; A61P 9/10

(52) U.S. Cl. CPC C12N 15/113; A61P 9/10; C12N 2310/113; C12N 2310/141; C12N 2310/3231

(57) ABSTRACT

Agents, compositions, and methods for the treatment of hypoxia and ischemia-related disorders. Antagonists of miR-106b, e.g., antisense oligonucleotides of miR-106b; duplexes comprising such antagonists and miR-93 nucleic acid molecules; and related compositions and methods.

### CROSS REFERENCE TO RELATED APPLICATIONS

The present application claims priority to U.S. Provisional Application No. 63/148,940, filed Feb. 12, 2021, the entire contents of which are hereby incorporated by reference for all purposes.

### SEQUENCE LISTING

The instant application contains a Sequence Listing which has been submitted electronically in ASCII format and is hereby incorporated by reference in its entirety. Said ASCII copy, created on Feb. 11, 2022, is named MPH-001WO_SL.txt and is 14 kb in size.

### BACKGROUND OF THE INVENTION

Peripheral arterial disease (PAD) affects approximately 8-12 million adults in the United States, having a significant impact on morbidity and mortality. PAD is characterized by reduced blood flow to the limbs resulting in ischemia with exercise or even at rest.

In response to tissue ischemia, a cascade of events can lead to sprouting of new blood vessels from existing capillaries in the vicinity of ischemic tissue to form new blood vessels. Such angiogenesis represents an adaptive mechanism to promote blood supply to ischemic tissue.

Therapeutic angiogenesis, the stimulation of growth of new blood vessels distal to the site of occlusion, represents a promising approach for creating a medical bypass to the ischemic tissue and improving perfusion in the ischemic tissue. A multitude of angiogenic growth factors have been exhaustively studied in both pre-clinical models of PAD and in clinical patients with PAD. Despite this, therapies to improve perfusion in peripheral ischemic tissue, e.g., ischemic limb, are yet lacking.

In the past decade, micro-RNAs have emerged as strong endogenous regulators of gene expression, particularly important in disease/injury states. Micro-RNAs (miRs) are 16-25 nucleotide non-coding RNAs that regulate gene expression, particularly in disease/injury states. Micro-RNAs typically work by targeting mRNA degradation or by direct translational repression, and they can regulate a single gene or entire pathways. Some micro-RNAs play crucial roles in developmental vasculogenesis and in tumor angiogenesis.

miRNAs are transcribed by RNA polymerase II (pol II) or RNA polymerase III and arise from initial transcripts, termed primary miRNA transcripts (pri-miRNAs), that are generally several thousand bases long. Pri-miRNAs are processed in the nucleus by the RNase Drosha into about 70- to about 100-nucleotide hairpin-shaped precursors (pre-miRNAs). Following transport to the cytoplasm, the hairpin pre-miRNA is further processed by Dicer to produce a double-stranded miRNA. The mature miRNA strand is then incorporated into the RNA-induced silencing complex (RISC), where it associates with its target mRNAs by base-pair complementarity.

However, information on the role of micro-RNAs in ischemia-induced angiogenesis such as in myocardial ischemia and PAD is limited.

There is a long felt need in the art for compositions and methods useful for enhancing endothelial cell and myocyte survival, for inducing angiogenesis, for treating ischemia, and for treating diseases, conditions, and disorders such as PAD.

### SUMMARY OF THE INVENTION

Provided herein are agents, compositions, and methods of treating or preventing a disease, disorder, injury, or condition associated with ischemia.

In one aspect, provided are nucleic acid duplexes comprising: (a) a miR-93 nucleic acid molecule; and (b) an antagonist of miR-106b.

In some embodiments, the duplex is an RNA:RNA duplex.

In some embodiments, the antagonist of miR-106b is an antisense oligonucleotide that is fully or partially complementary to at least a portion of miR-106b.

In some embodiments, the duplex comprises a miR-93 RNA comprising the sequence AAAGUGCUGUUCGUGCAGGUAG (SEQ ID NO: 3) or a miR-93 RNA comprising the sequence CAAAGUGCUGUUCGUGCAGGUAG (SEQ ID NO: 4).

In some embodiments, the duplex comprises an antisense oligonucleotide of miR-106b, the antisense oligonucleotide comprising the sequence

(SEQ ID NO: 6)
AUCUGCACUGUCAGCACUUUA.

In one aspect, provided are antagonists of miR-106b expression, level, or activity.

In some embodiments, the antagonist is an antisense oligonucleotide that is fully or partially complementary to at least a portion of miR-106b.

In some embodiments, the miR-106b is human miR-106b-5p comprises the sequence UAAAGUGCUGACAGUGCAGAU (SEQ ID NO: 1).

In some embodiments, the miR-106b is human miR-106b-3p comprises the sequence CCGCACUGUGGGUACUUGCUGC (SEQ ID NO: 2).

In some embodiments, the antagonist comprises DNA.

In some embodiments, the antagonist comprises RNA.

In some embodiments, the antagonist is an antagomir miR-106b.

In some embodiments, the antisense oligonucleotide comprises one or more nucleotide analogs. For example, the one or more nucleotide analogs may comprise a locked nucleic acid (LNA).

In some embodiments, the antisense oligonucleotide is capable of forming a duplex with a mature miR-106b molecule, the duplex having a melting temperature (Tₘ) of at least about 60° C.

In some embodiments, the antisense oligonucleotide is capable of forming a duplex with another single stranded RNA molecule. In some embodiments, the other single stranded RNA molecule is a miR-93 RNA molecule.

In some embodiments, the duplex of the antisense oligonucleotide and the other single stranded RNA molecule has a Tₘ of less than about 65° C., less than about 60° C., less than about 55° C., less than about 50° C., less than about 45° C., less than about 40° C., less than about 37° C., less than about 35° C., less than about 30° C., or less than about 25° C.

In some embodiments, the antagonist is an antisense oligonucleotide that is fully or partially complementary to at least a portion of miR-106b; the antisense oligonucleotide is capable of forming a duplex with a mature miR-106b molecule; the antisense oligonucleotide is capable of forming a duplex with another single stranded RNA molecule; and the Tₘ of a duplex formed by the antisense oligonucleotide with a mature miR-106b molecule is greater than the Tₘ of a duplex formed by the antisense oligonucleotide with the other single stranded RNA molecule. In some such embodiments, the other single stranded RNA molecule is an miR-93 RNA molecule. In some embodiments, the miR-93 RNA molecule comprises the sequence AAAGUGCUGUUCGUGCAGGUAG (SEQ ID NO: 3) or the sequence CAAAGUGCUGUUCGUGCAGGUAG (SEQ ID NO: 4).

In some embodiments, the antagonist is encoded by an isolated nucleic acid, or vector comprising the isolated nucleic acid. In some embodiments, the vector is an expression vector selected from an miRNA expression vector or AAV expression vector. For example, the expression vector may be an miRNA expression vector. In some embodiments, the isolated nucleic acid is operably-linked to a cell-specific promoter.

In some embodiments, the antagonist or nucleic acid duple disclosed herein is encapsulated within a lipid vehicle.

In one aspect, provided are pharmaceutical compositions comprising (a) an effective amount of the nucleic acid duplex of any one of claim 1-5 or 26 or the antagonist of any one of claims 6-26; and (b) a pharmaceutically acceptable carrier.

In some embodiments, pharmaceutical compositions further comprise an additional therapeutic agent. For example, the additional therapeutic agent may comprise an anti-ischemia agent.

In some embodiments, the effective amount is effective to decrease expression of at least one cell cycle pathway gene in an endothelial or muscle cell of a subject who is administered the pharmaceutical composition. For example, said cell cycle pathway genes may be selected from the group consisting of E2F-1 and p53. In some embodiments, the expression is in skeletal muscle cells.

In some embodiments, the effective amount is effective to enhance perfusion recovery in a subject who is administered the pharmaceutical composition.

In some embodiments, the effective amount is effective to enhance angiogenic response to ischemia in a subject who is administered the pharmaceutical composition.

In some embodiments, the effective amount is effective to stimulate cell proliferation, for example, cell proliferation comprising proliferation of endothelial cells or muscle cells.

In some embodiments, the effective amount is effective to increase capillary density in a subject who is administered the pharmaceutical composition.

In some embodiments, the effective amount is effective to inhibit apoptosis of one or more cells in a subject who is administered the pharmaceutical composition. For example, in some embodiments, the apoptosis is hypoxia-induced apoptosis.

In some embodiments, the pharmaceutical composition is formulated for administration by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

In some embodiments, said administration is intramuscular.

In one aspect, provided are methods of treating or preventing a disease, disorder, injury, or condition associated with ischemia, said method comprising administering to a subject in need thereof a pharmaceutical composition as disclosed herein.

In some embodiments, methods further comprise administering to the subject an additional therapeutic agent, e.g., an anti-ischemia agent

In some embodiments, the effective amount is effective to decrease expression of, or attenuate ischemia-induced upregulation of, at least one cell cycle pathway gene in an endothelial or muscle cell of the subject. In some embodiments, the cell cycle pathway genes are selected from the group consisting of E2F-1 and p53. In some embodiments, the expression is in skeletal muscle cells.

In some embodiments, the effective amount is effective to enhance perfusion recovery in the subject.

In some embodiments, the effective amount is effective to enhance angiogenic response to ischemia in the subject.

In some embodiments, the effective amount is effective to stimulate cell proliferation.

In some embodiments, the cell proliferation comprises proliferation of endothelial cells or muscle cells.

In some embodiments, the effective amount is effective to increase capillary density in the subject.

In some embodiments, the effective amount is effective to inhibit apoptosis of one or more cells in the subject. In some embodiments, the apoptosis is hypoxia-induced apoptosis.

In some embodiments, administration is by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

In some embodiments, administration is intramuscular.

In some embodiments, the subject is a human.

In some embodiments, the ischemia is selected from the group consisting of vascular ischemia, muscular ischemia, peripheral arterial disease, ischemia reperfusion injury, ischemia associated with trauma, and brain ischemia.

In some embodiments, the ischemia is peripheral arterial disease.

### DETAILED DESCRIPTION OF CERTAIN EMBODIMENTS OF THE INVENTION

### Abbreviations

bw—body weight
C2C12—an immortalized mouse muscle cell line
FDR—false discovery rate
GA—gastrocnemius muscle
GF—growth factor
GSEA—Gene Set Enrichment Analysis
HLI—hind-limb ischemia
HSS—hypoxia and serum starvation
HUVEC—human umbilical vein endothelial cell
IGA—ischemic gastrocnemius muscle
LNA—locked nucleic acid
LSGS—low serum growth supplement
miR—micro-RNA (also referred to as miRNA)
miRNA—micro-RNA (also referred to as miR)
NGA—non-ischemic gastrocnemius muscle
PAD—peripheral arterial disease
PremiR—a precursor miRNA
RISC—RNA-induced silencing complex
SEM—standard error of the mean
TA—tibialis anterior
VEGF—vascular endothelial growth factor

### Definitions

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

An “antagonist”, as used herein, refers to an agent which inhibits the level or biological activity of a target agent. For example, an “antagonist” of miR-106b refers to an agent that reduces the level or activity of miR-106b in vitro, ex vivo, or in vivo.

The term “antagomir” refers to a small RNA or DNA (or chimeric) molecule to antagonize endogenous small RNA regulators like microRNA (miRNA). These antagonists bear complementary nucleotide sequences for the most part, which means that antagomirs should hybridize to, e.g., the mature microRNA (miRNA), or a pre-miRNA precursor of the mature microRNA. They prevent other molecules from binding to a desired site on an mRNA molecule and are used to silence endogenous microRNA (miR). Antagomirs are therefore designed to, e.g., block biological activity, reduce expression, levels, or activity of these post-transcriptional molecular modulators of response to injury.

As used herein, the term “attach”, or “attachment”, or “attached”, or “attaching”, used herein interchangeably with “bind”, or “binding” or “binds” or “bound” refers to any physical relationship between molecules that results in forming a stable complex, such as a physical relationship between a ligand, such as a peptide or small molecule, with a “binding partner” or “receptor molecule.” The relationship may be mediated by physicochemical interactions including, but not limited to, a selective noncovalent association, ionic attraction, hydrogen bonding, covalent bonding, van der Waals forces or hydrophobic attraction.

“Complementary” as used herein refers to the broad concept of subunit sequence complementarity between two nucleic acids, e.g., an RNA sequence and an antisense oligonucleotide to the RNA sequence. When a nucleotide position in both of the molecules is occupied by nucleotides normally capable of base pairing with each other, then the nucleic acids are considered to be complementary to each other at this position. Thus, two nucleic acids are complementary to each other when a substantial number (e.g., at least 50%) of corresponding positions in each of the molecules are occupied by nucleotides which normally base pair with each other (e.g., A:T (or A:U for duplexes comprising RNA) and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, e.g., at least about 50%, at least about 55%, at least about 60%, at least about 65%, at least about 70%, at least about 75%, at least about 80%, at least about 85%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

An “effective amount” or a “therapeutically effective amount” (or “prophylactically effective amount”) is used herein to refer that amount sufficient to effect beneficial or desired results, such as increased angiogenesis or particular desired clinical results. Therefore an “effective amount” or “therapeutically effective amount” depends upon the context in which it is being applied.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of one or more symptoms of the disease, diminishment of any direct or indirect pathological consequences of the disease, decreasing the rate of disease progression, amelioration or palliation of the disease state, and improved prognosis.

As used herein, the term “therapeutically effective amount” or “effective amount” refers to an amount of an agent or pharmaceutical composition provided herein that, when administered to a subject, is effective to treat a disease or disorder.

The term “in vitro” refers to processes that occur outside a living organism. Such term encompasses the term “in situ” and “ex vivo”.

The term “in situ” refers to processes that occur in a living cell growing separate from a living organism, e.g., growing in tissue culture.

The term “ex vivo” refers to processes that occur in a tissue sample, generally wherein cytoarchitecture of the tissue is preserved.

The term “in vivo” refers to processes that occur in a living organism.

The term “mammal” as used herein includes both humans and non-humans and include but is not limited to humans, non-human primates, canines, felines, murines, bovines, equines, and porcines.

A “subject” of analysis, diagnosis, or treatment is an animal. Such animals include mammals, preferably a human.

As used herein, a “subject in need thereof” is a patient, animal, mammal, or human, who may benefit from the method of this invention.

As used herein, the term “subject at risk for PAD” refers to a subject with one or more risk factors for developing PAD. Risk factors include, but are not limited to, gender, age, genetic predisposition, environmental expose, and previous incidents of PAD, and lifestyle.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

It must be noted that, as used in the specification and the appended claims, the singular forms “a,” “an” and “the” include plural referents unless the context clearly dictates otherwise.

### Overview

When compared to controls, higher levels of miR-106b significantly decreases endothelial branching in an in vitro angiogenesis assay performed under PAD relevant conditions. (Ganta et al., Circulation. 2017; 135:2403-2425, which is hereby incorporated by reference in its entirety.)

Disclosed herein are, among other things, antagonists of miR-106b expression, levels, or activity. Such agents are useful to treat and prevent hypoxia, ischemia, and other injuries, diseases, disorders and conditions associated with ischemia such as peripheral arterial disease and myocardial ischemia. Without wishing to be bound by theory, such antagonists may attenuate or block miR-106b-mediated inhibition of angiogenesis, which would be useful in aiding recovery from an ischemic event.

Nucleic Acid Duplexes and miR-106b Antagonists

Provided herein are nucleic acid duplexes comprising: (a) a miR-93 nucleic acid molecule; and (b) an antagonist of miR-106b, such as any antagonist described herein.

The term “duplex” as used herein in reference to a nucleic acid duplex is used in accordance with its meaning in the art and may refer to duplexes comprising DNA strands, RNA strands, or both DNA and RNA. In some embodiments, the duplex is an RNA:RNA duplex. As is understood by a person of skill the art, at least a portion of each strand in the duplex is substantially complementary to at least a portion of the other strand, and the degree of complementarity may vary and may, but need not, be 100%. For example, some duplexes contain at least one, at least two, at least three, at least four, or at least five mismatches between the strands within a region of complementarity or throughout the entire duplex.

In some embodiments, the antagonist of miR-106b is an antisense oligonucleotide that is fully or partially complementary to at least a portion of miR-106b. In some embodiments, the antisense oligonucleotide comprises the sequence AUCUGCACUGUCAGCACUUUA (SEQ ID NO: 6) or a sequence having at least 80%, at least 85%, at least 90%, or at least 95%, identical thereto. In some embodiments, the antisense oligonucleotide has the sequence of SEQ ID NO: 6 or a sequence having at least 80%, at least 85%, at least 90%, or at least 95%, identical thereto.

Examples miR-93 nucleic acid molecules include, but are not limited to, those disclosed in U.S. Pat. No. 9,845,465, the entire contents of which are herein incorporated by reference.

In some embodiments, the miR-93 nucleic acid molecule is a miR-93 RNA, such as a miR-93 comprising the sequence AAAGUGCUGUUCGUGCAGGUAG (SEQ ID NO: 3) (or a sequence having at least 80%, at least 85%, at least 90%, or at least 95%, identical thereto) or a miR-93 RNA comprising the sequence CAAAGUGCUGUUCGUGCAGGUAG (SEQ ID NO: 4) (or a sequence having at least 80%, at least 85%, at least 90%, or at least 95%, identical thereto). In some embodiments, the miR-93 RNA is an oligonucleotide having the sequence of SEQ ID NO: 3 or SEQ ID NO: 4 (or a sequence having at least 80%, at least 85%, at least 90%, or at least 95%, identical to either SEQ ID NO: 3 or SEQ ID NO: 4).

In some embodiments, the antagonist of miR-106b is an antagonist of mammalian miR-106b. In some embodiments, the antagonist of miR-106b is an antagonist of human miR-106b.

In some embodiments, the antagonist of human miR-106b is an antagonist of hsa-miR-106b-5p. The mature sequence of hsa-miR-106b-5p, as noted in miRBase, is:

(SEQ ID NO: 1)
UAAAGUGCUGACAGUGCAGAU.

In alternative embodiments, the antagonist of human miR-106b is an antagonist of hsa-miR-106b-3p. The mature sequence of hsa-miR-106b-3p, as noted in miRBase, is

(SEQ ID NO: 2)
CCGCACUGUGGGUACUUGCUGC.

In some embodiments, the antagonist of miR-106b is an antisense oligonucleotide comprising a sequence that is fully or partially complementary to a portion of mature miR-106b such that the antisense oligonucleotide binds to miR-106b. In some embodiments, the antisense oligonucleotide sequence is fully or partially complementary to a portion of mature miR-106b such that the antisense oligonucleotide binds to miR-106b under PAD relevant conditions. See, e.g., Ganta et al., Circulation. 2017; 135:2403-2425, which is hereby incorporated by reference in its entirety. Such antisense oligonucleotides may be referred to herein as “targeting” a miR-106b nucleic acid or an “antisense oligonucleotide of” miR-106b. In some embodiments, the antisense oligonucleotide comprises a sequence that is at least 85%, 90%, or 95% complementary, or 100% complementary, to the portion of mature miR-106b. In preferred embodiments, the antisense oligonucleotide comprises a sequence that is 100% complementary to the portion of mature miR-106b. In some embodiments, the antagonist of miR-106b comprises an antisense oligonucleotide comprising a sequence that is at least 80%, at least 85%, at least 90%, or at least 95% identical to at least a portion of the sequence of SEQ ID NO: 6 or SEQ ID NO: 7, shown in Table 1. In some embodiments, the antagonist of miR-106b comprises a sequence that is 100% identical to at least a portion of the sequence of SEQ ID NO: 6 or SEQ ID NO: 7. In some embodiments, the portion comprises at least 7, 8, 9, 10, 11, 12, 13, 14, 15, 16, 17, 18, 19, 20, or 21 contiguous nucleotides of SEQ ID NO: 6 or 7. In some embodiments, the portion comprises all of SEQ ID NO: 6 or 7.

In some embodiments, the antagonist of miR-106b comprises a sequence that is 100% identical to a portion comprising at least 7, 8, 9, 10, 11, 12, 13, 14, 15, 16, 17, 18, 19, 20, or 21 contiguous nucleotides of SEQ ID NO: 6 or 7.

TABLE 1
Exemplary sequences of antisense
oligonucleotides of miR-106b antagonists

| SEQ ID NO: | Sequence |
|---|---|
| 6 | AUCUGCACUGUCAGCACUUUA |
| 7 | GCAGCAAGUACCCACAGUGCGG |

In a preferred aspect, the antagonist is an antisense oligonucleotide of miR-106b-5p.

In one aspect, the antagonist is an antisense oligonucleotide of miR-106b-3p.

In some embodiments, the portion of mature miR-106b comprises at least 7, 8, 9, 10, 11, 12, 13, 14, 15, 16, 17, 18, 19, 20, 21, or all 22 contiguous nucleotides present in the miR-106b sequence. In preferred embodiments, the portion of mature miR-106b comprises all 22 contiguous nucleotides present in the hsa-miR-106b-5p sequence.

In some embodiments, the antisense oligonucleotide comprises a sequence that is 100% complementary to all 22 contiguous nucleotides present in the hsa-miR-106b-5p sequence.

In some embodiments, the portion of mature miR-106b comprises the miR-106b seed region. Accordingly, in some embodiments the antisense oligonucleotide comprises a region which is fully or partially complementary to the miR-106b seed region. In some embodiments, the antisense oligonucleotide comprises a region which is fully complementary to the miR-106b seed region.

In some embodiments, the antisense oligonucleotide comprises a sequence which is fully complementary to miR-106b as measured across the length of the antisense oligonucleotide. In some embodiments, the antisense oligonucleotide comprises a sequence which is fully complementary to miR-106b as measured across the length of miR-106b. In some embodiments, the antisense oligonucleotide is complementary to a corresponding region of the microRNA across the length of the antisense oligonucleotide. In some embodiments, the 3′ nucleoside of the oligomer is complementary to (i.e. aligns with) the first, second, third or fourth 5′ nucleotides of miR-106b. In an embodiment, the 3′ nucleoside of the oligomer aligns with the second 5′ nucleotide of miR-106b.

In some embodiments, the antisense oligonucleotide is mostly complementary to miR-106b across the length of the oligomer. For example, the antisense oligonucleotide may comprise one mismatch to the corresponding region of miR-106b. For example, the antisense oligonucleotide may comprise two mismatches to the corresponding region of miR-106b.

In some embodiments, the antisense oligonucleotide comprises DNA. In some embodiments, the antisense oligonucleotide comprises RNA.

In some embodiments, the antisense oligonucleotide is capable of forming a duplex with miR-106b. In some embodiments, the duplex has a melting temperature (Tₘ). The Tₘ can be, e.g., least about 60° C., about 65° C., about 70° C., or higher. In some embodiments, the Tₘ is between about 60° C. and about 90° C., between about 65° C. and about 85° C., or between about 70° C. and about 80° C.

In some embodiments, the antisense oligonucleotide binds to miR-106b to a sufficient degree to reduce miR-106b-mediated inhibition of angiogenesis relevant to ischemia recovery, but not to an excessive degree as to reduce miR-106b-mediated pathway activity unrelated to ischemia recovery. In particular embodiments, the antisense oligonucleotide binds to miR-106b to a sufficient degree to treat a disease, disorder, or condition associated with ischemia but not to an excessive degree as to cause unwanted side effects.

In some embodiments, the antisense oligonucleotide is capable of forming a duplex with another single stranded RNA molecule (other than miR-106b, e.g., other than hsa-miR-106b-5p). In some embodiments, a duplex that is formed with the other single-stranded RNA is less stable than a duplex that is formed with miR-106b. In some embodiments, the Tₘ of the duplex with the other single stranded RNA nucleic molecule is less than about 60° C., less than about 55° C., less than about 50° C., less than about 45° C., less than about 40° C., less than about 37° C., less than about 35° C., less than about 30° C., or less than about 25° C. In some embodiments, the Tₘ of the duplex with the other single stranded RNA nucleic molecule is at least 25° C., at least 30° C., at least 35° C., at least 37° C., at least 40° C., at least 45° C., at least at least 50° C., or at least 55° C.

In some embodiments, the Tₘ of a duplex formed by the antisense oligonucleotide with a mature miR-106b molecule is greater than the Tₘ of a duplex formed by the antisense oligonucleotide with the other single stranded RNA molecule.

In some embodiments, the antisense oligonucleotide does not form a duplex with another single-stranded RNA molecule (other than miR-106b, e.g., other than hsa-miR-106b-5p), under physiological conditions or under PAD-relevant conditions. In some embodiments, the antisense oligonucleotide does not form a duplex with another single-stranded RNA molecule (other than miR-106b, e.g., other than hsa-miR-106b-5p) in vivo. In some embodiments, the other single-stranded RNA molecule is miR-93.

The Tₘ of a given duplex may be determined by a Tₘ assay. In an exemplary Tₘ assay provided herein, the antisense oligonucleotide and RNA target duplexes are diluted to 3 mM in 500 ml RNase-free water and mixed with 500 ml 2×Tₘ-buffer (200 mM NaCl, 0.2 mM EDTA, 20 mM Na phosphate, pH 7.0). The solution is heated to 95° C. for 3 min and then allowed to anneal in room temperature for 30 min. The duplex melting temperatures (Tₘ) is measured on a Lambda 40 UVA IS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

The length of the antisense oligonucleotide can be, e.g., from about seven to about 30 nt, such as about seven to about 26 or about eight to about 25, such as about seven, about eight, about nine, about 10, about 11, about 12, about 13, about 14, about 15, about 16, about 17, about 18, about 19, about 20, about 21, about 22, about 23, about 24, about 25 nucleotides in length, such as about 10 to about 22 nucleotides in length. In some embodiments, the length is 21 or 22 nucleotides.

In an aspect, the antisense oligonucleotide is a miR-106b antagomir.

Antagomirs (also referred to herein as antimiRs) generally refer to oligomers which consist or comprise of a contiguous nucleotide sequence which is fully complementary to, or essentially complementary to (i.e. may comprise one or two mismatches), to a microRNA sequence, or a corresponding subsequence thereof. In this regard, it is considered that the antimiR may be comprise a contiguous nucleotide sequence which is complementary or essentially complementary to the entire mature microRNA, or the antimiR may be comprise a contiguous nucleotide sequence which is complementary or essentially complementary to a sub-sequence of the mature microRNA or pre-microRNA. The sub-sequence (and therefore the corresponding contiguous nucleotide sequence can be at least five, six, seven, or eight nucleotides in length, such as between five and 25 nucleotides, such as five, six, seven, eight, nine, 10, 11, 12, 13, 14, 15, 16, 17, 18, 19, 20, 21, 22, 23, 24, or 25 nucleotides in length, such as between 10 and 17 or between 10 and 16 nucleotides, such as between 12 and 15 nucleotides. Numerous designs of antimiRs have been suggested, and typically, in antimiRs for therapeutic use, the contiguous nucleotide sequence of the antimiR comprises one or more nucleotide analogues.

In some embodiments, the antimiR may has a gapmer structure. However, as explained in WO2007/112754 and WO2007/112753, both of which are hereby incorporated by reference in their entirety, other designs may be preferable, such as mixmers, or totalmers.

WO2007/112754 and WO2007/112753, both hereby incorporated by reference in their entirety, provide antimiR oligomers and antimiR oligomer designs where the oligomers are complementary to mature microRNA

In some embodiments, a subsequence of the antimiR corresponds to the miRNA seed region. In some embodiments, the first or second 3′ nucleobase of the oligomer corresponds to the second 5′ nucleotide of the microRNA sequence.

In some antimiR embodiments, nucleobase units one to six (inclusive) of the oligomer as measured from the 3′ end the region of the oligomer are complementary to the microRNA seed region sequence.

In some antimiR embodiments, nucleobase units one to seven (inclusive) of the oligomer as measured from the 3′ end the region of the oligomer are complementary to the microRNA seed region sequence.

In some antimiR embodiments, nucleobase units two to seven (inclusive) of the oligomer as measured from the 3′ end the region of the oligomer are complementary to the microRNA seed region sequence.

In some embodiments, the antimiR oligomer comprises at least one nucleotide analogue unit, such as at least one LNA unit, in a position which is within the region complementary to the miRNA seed region. The antimiR oligomer may, in some embodiments comprise at between one and six or between one and seven nucleotide analogue units, such as between one and six and one and seven LNA units, in a position which is within the region complementary to the miRNA seed region.

In some embodiments, the antimiR comprises a contiguous nucleotide sequence which is complementary to a seed region of miR-106b, and wherein at least 80%, such as at least 85%, at least 90%, at least 95%, or 100% of the nucleotides are LNA.

In some embodiments, the antimiR comprises a contiguous nucleotide sequence which is complementary to a seed region of miR-106b, and wherein at least 80% of the nucleotides are LNA, and wherein at least 80%, at least 85%, at least 90%, at least 95%, or 100% of the internucleotide bonds are phosphorothioate bonds.

In some embodiments, the contiguous nucleotide sequence of the antimiR comprises no more than a single mismatch when hybridizing to the target sequence. In some embodiments, the contiguous nucleotide sequence comprises no more than two mismatches when hybridizing to the target sequence. In some embodiments, the contiguous nucleotide sequence comprises no mismatches when hybridizing to the target sequence.

In determining the degree of “complementarity” between oligomers of the disclosure (or regions thereof) and the target region of the nucleic acid, such as those disclosed herein, the degree of “complementarity” (also, “homology” or “identity”) may be expressed as the percentage identity (or percentage homology) between the sequence of the oligomer (or region thereof) and the sequence of the target region (or the reverse complement of the target region) that best aligns therewith. The percentage is calculated by counting the number of aligned bases that are identical between the two sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the invention and the target region.

As used herein, the terms “homologous” and “homology” are interchangeable with the terms “identity” and “identical.”

The terms “corresponding to” and “corresponds to” refer to the comparison between the nucleotide sequence of the oligomer (i.e. the nucleobase or base sequence) or contiguous nucleotide sequence (a first region) and the equivalent contiguous nucleotide sequence of a further sequence selected from either (i) a sub-sequence of or (ii) the reverse complement of the nucleic acid target. Nucleotide analogues are compared directly to their equivalent or corresponding nucleotides. A first sequence which corresponds to a further sequence under i) or ii) typically is identical to that sequence over the length of the first sequence (such as the contiguous nucleotide sequence) or, as described herein may, in some embodiments, is at least 80% homologous to a corresponding sequence, such as at least 85%, at least 90%, at least 91%, at least 92%, at least 93%, at least 94%, at least 95%, at least 96% homologous, such as 100% homologous (identical).

### Nucleotide Analogues

In some embodiments, the antisense oligonucleotide comprises one or more nucleoside analogues. In some embodiments, the antisense oligonucleotide comprises one or more nucleotide analogues.

Exemplary nucleoside analogue units include, but are not limited to, 2′-O-alkyl-RNA units, 2′-OMe-RNA units, 2′-amino-DNA units, 2′-fluoro-DNA units, LNA units, PNA units, HNA units, 2′-FANA, 2′-(3-hydroxy)propyl, and 2′-fluoro-DNA units, and/or other (optionally) sugar modified nucleoside analogues such as morpholino, peptide nucleic acid (PNA), CeNA, unlinked nucleic acid (UNA), hexitol nucleic acid (HNA). bicyclo-HNA (see, e.g., WO2009/100320, which is hereby incorporated by reference in its entirety), and INA units. In some embodiments, the one or more nucleoside analogues increase the affinity of the first region for its target nucleic acid (or a complementary DNA or RNA sequence). Various nucleoside analogues are disclosed in Freier & Altmann; Nucl. Acid Res., 1997, 25, 4429-4443 and Uhlmann; Curr. Opinion in Drug Development, 2000, 3(2), 293-213, which are hereby incorporated by reference.

The terms “LNA unit”, “LNA monomer”, “LNA residue”, “locked nucleic acid unit”, “LNA”, “bicyclic nucleic acid”, “BNA”, “locked nucleic acid monomer” or “locked nucleic acid residue”, are used interchangeably herein to refer to a bicyclic nucleoside analogue. In some embodiments, the LNA comprises a bridge (or biradical) linking the second and forth carbon of the ribose ring, (C4*-C2* bridge or biradical). The presence of the biradical between the 2nd and 4th carbon locks the ribose into a 3′ endo-(north) confirmation. LNA units are described in WO 99/14226, WO 00/56746, WO 00/56748, WO 01/25248, WO 02/28875, WO 03/006475 and WO 03/095467, which are hereby incorporated by reference in their entirety.

In some embodiments, the LNA unit or units are independently selected from the group consisting of oxy-LNA, thio-LNA, and amino-LNA, in either of the D-β and L-α configurations or combinations thereof.

In some embodiments, the LNA comprises an ENA nucleobase.

In some embodiments, the LNA comprises beta D oxy-LNA.

In some embodiments, the LNA comprises alpha-L amino LNA.

2′ substituted oligomers, e.g., fully 2′ OME oligomers, are described in WO05/013901, WO07/027775, WO07027894, each of which are hereby incorporated by reference in their entirety. In some embodiments, the first region of the oligomer may comprise of 2′ substituted nucleosides. WO07/027775 also refers to MOE, LNA, DNA mixmers for use in targeting microRNAs.

The terms “corresponding nucleotide analogue” and “corresponding nucleotide” are intended to indicate that the nucleotide in the nucleotide analogue and the naturally occurring nucleotide are identical. For example, when the 2-deoxyribose unit of the nucleotide is linked to an adenine, the “corresponding nucleotide analogue” contains a pentose unit (different from 2-deoxyribose) linked to an adenine.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell.

Examples of suitable nucleotide analogues are described in WO2007/031091, which is hereby incorporated by reference in its entirety. Other nucleotide analogues which may be used in an antisense oligonucleotide disclosed herein include tricyclic nucleic acids. Exemplary tricyclic nucleic acids are described in WO2013154798 and WO2013154798, each of which are hereby incorporated by reference in their entirety.

In some embodiments, nucleosides comprise a chemically modified ribofuranose ring moiety.

In some embodiments, the antisense oligonucleotide comprises one or two LNA units. In some embodiments, the one or two LNA units are in positions three to eight, counting from the 3′ end. Such positioning can be advantageous for the stability of the A-helix formed by the oligo:microRNA duplex, a duplex resembling an RNA:RNA duplex in structure.

### Vectors

In some embodiments, the miR-106b antagonist is encoded in a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In some embodiments, an expression vector comprises a promoter “operably linked” to a polynucleotide encoding the miR-106b antagonist. The phrase “operably linked” or “under transcriptional control” as used herein means that the promoter is in the correct location and orientation in relation to a polynucleotide to control the initiation of transcription by RNA polymerase and expression of the polynucleotide. The polynucleotide encoding the miR-106b antagonist may encode the primary-microRNA sequence, the precursor-microRNA sequence, or the mature the miR-106b antagonist sequence.

In some embodiments, the expression vector is a viral vector such as one derived from adenoviruses, adeno-associated viruses (AAV), or retroviruses, including lentiviruses such as the human immunodeficiency (HIV) virus. In some embodiments, the AAV is AAV8 or AAV9.

In one aspect, the vector is an AAV (adeno-associated virus) vector. In some embodiments, a recombinant AAV vector of the disclosure is useful for targeting muscle preferentially over other tissues. In some embodiments, a recombinant AAV vector of the disclosure is useful for increasing expression of a gene of interest preferentially in muscle. In some embodiments, compositions and methods disclosed herein encompass targeting and transducing muscle with an AAV vector. Methods may comprise administering to a subject a pharmaceutical composition comprising an effective amount of a recombinant adeno-associated viral (AAV) vector comprising a regulatory element. The regulatory element generally comprises at least one promoter element and optionally at least one enhancer element. An enhancer and promoter are typically operably linked. The recombinant AAV vector also may optionally comprise at least one gene operably linked to a promoter element. The AAV may, in some embodiments, comprise the entire AAV genome, or a homolog or fragment thereof, such as the capsid of the particular AAV. However, it should be noted that the entire AAV genome may not be useful or required in some situations because of a need to make the vector replication-deficient and/or to insert genes of interest such as therapeutic genes.

The regulatory elements and the gene of interest may also be substituted with active fragments, modifications, or homologs thereof. In one aspect, the recombinant AAV vector preferentially targets skeletal muscle.

A recombinant AAV vector can be prepared for use in knocking down specific genes in muscle with siRNA or miRNA expressed from an AAV vector of the disclosure.

Other useful vectors, nucleic acids, and proteins or homologs and fragments thereof may be useful with the practice of the disclosure, including but not limited to AAV-9 (e.g., NCBI Accession number AX753250; SEQ ID NO: 8) and AAV-8 (e.g., NCBI Accession number NC006261; SEQ ID NO: 9).

Due to the payload constraints of AAV, in some embodiments, a cDNA may be used. In one aspect, additional introns and sequences can be introduced. In one aspect, the cap gene of the AAV is used and not the entire AAV genomic DNA.

Other methods and vectors are known in the art which could also be used to practice the methods of the present invention, including those in Souza et al. (e.g., U.S. Pat. Pub. No. 2011/0212529, published Sep. 1, 2011).

Although AAVs such as AAV9 and AAV8 may target some tissues with higher specificity than other tissues, the use of tissue or cell specific enhancers and promoters as part of the vector can help to ensure that the genes of interest are expressed in the desired cell or tissue. In some embodiments, the desired cell or tissue comprises skeletal and/or striated muscle cells. In some embodiments, the desired cell or tissue comprises cardiac muscle cells.

A more detailed description and use of AAVs can be found, e.g., in U.S. Pat. Pub. No. US 2013/0136729 (French and Annex, U.S. patent application Ser. No. 13/673,351), the entirety of which is incorporated by reference herein.

In some embodiments, the vector comprises a cardiac troponin-T gene promoter or an essential proximal promoter element thereof. Exemplary cardiac troponin-T promoters and essential elements are describe in U.S. Pat. No. 5,266,488, the contents of which is hereby incorporated by reference in its entirety. In some embodiments, the vector comprises a muscle creatine kinase promoter or an essential proximal promoter element thereof. In some embodiments, the vector comprises a desmin (DES) promoter or an essential proximal promoter element thereof. In some embodiments, the vector comprises a tissue-specific enhancer.

In some embodiments, the expression vector is optimized for sustained expression of a transgene in muscle tissue. Another object of this invention is to provide enhancer/promoter combinations that can direct sustained and appropriate expression levels in various expression systems.

In some embodiments, the expression vector comprises combinations of minimal sequences from muscle-specific promoters and muscle-specific enhancers to create chimeric regulatory elements that drive transcription of a transgene in a sustained fashion. A minimal sequence is one which maintains the function of interest, although possibly somewhat less than the full sequence of interest.

The present disclosure further provides cells transfected with a nucleic acid containing an enhancer/promoter combination of the present disclosure.

Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, term “regulatory elements” is used interchangeably with “regulatory sequences” and refers to promoters, enhancers, and other expression control elements, or any combination of such elements.

### Functional Impact

In some embodiments, the antagonist of miR-106b, e.g., the antisense oligonucleotide to miR-106b, reduces mature miR-106b expression, biological activity, or both expression and biological activity.

Mature miR-106b expression can be determined according to any known methods in the art, including but not limited to: qPCR, e.g., real-time qPCR, and microarray. Mature miR-106b expression can be determined in vitro, e.g., using cell culture models, ex vivo, or in vivo.

In some embodiments, the antagonist of miR-106b, e.g., the antisense oligonucleotide to miR-106b, reduces mature miR-106b expression by at least 10%, at least 20%, at least 30%, at least 40%, at least 50%, at least 55%, at least 60%, at least 65%, at least 70%, at least 75%, at least 80%, at least 85%, at least 90%, or at least 95%. In some embodiments, the antagonist of miR-106b, e.g., the antisense oligonucleotide to miR-106b, reduces mature miR-106b expression by at least 10%, at least 20%, at least 30%, at least 40%, or at least 50%, e.g., between 10 and 100%, between 20 and 90%, between 30 and 80%, between 40 and 70%, or between 50 and 60%. In some embodiments, the antagonist of miR-106b, e.g., the antisense oligonucleotide to miR-106b, reduces mature miR-106b expression by at least 60%, at least 65%, at least 70%, at least 75%, at least 80%, at least 85%, at least 90%, or at least 95%.

In some embodiments, the antagonist of miR-106b (e.g., antisense oligonucleotide to miR-106b) reduces miR-106b pathway activity.

In some embodiments, the antagonist of miR-106b (e.g., antisense oligonucleotide to miR-106b) promotes angiogenesis under PAD relevant conditions. Angiogenesis under PAD relevant conditions can be determined by any means known in the art.

In some embodiments, the antagonist of miR-106b (e.g., antisense oligonucleotide to miR-106b) attenuates caspase-9 expression or attenuates hypoxia-induced caspase-9 expression. In some embodiments, the antagonist of miR-106b attenuates hypoxia-induced caspase-9 expression by at least about 5%, by at least about 10%, by at least about 15%, by at least about 20%, or by about more than 20%. In some embodiments, the antagonist of miR-106b attenuates hypoxia-induced caspase-9 expression by about 5 to about 15%. In some embodiments, the antagonist of miR-106b attenuates hypoxia-induced caspase-9 expression by at least about 10%, e.g., about 10%.

In some embodiments, the antagonist of miR-106b (e.g., antisense oligonucleotide to miR-106b) attenuates ischemia-induced upregulation of, or reduces expression of, one or more genes in endothelial or muscle cells. In some embodiments, the one or more genes are genes of the cell cycle pathway. In some embodiments, the one or more genes of the cell cycle pathway are selected from p53 and E2F-1. In some embodiments, the expression of the one or more genes is assessed in the cells following an ischemic event.

In some embodiments, the antagonist of miR-106b (e.g., antisense oligonucleotide to miR-106b) attenuates ischemia-induced upregulation, or reduces expression of, p53. In some embodiments, p53 mRNA expression is reduced by about 20% to at about 40%. In some embodiments, p53 mRNA expression is reduced by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, p53 protein expression is reduced by about 10 to about 30%. In some embodiments, p53 protein expression is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

In some embodiments, the antagonist of miR-106b (e.g., antisense oligonucleotide to miR-106b) attenuates ischemia-induced upregulation of, or reduces expression of, E2F-1. In some embodiments, E2F-1 mRNA expression is reduced by about 60% to about 80%. In some embodiments, E2F-1 protein expression is reduced by about 10% to about 30%.

In some embodiments, the antagonist of miR-106b (e.g., antisense oligonucleotide to miR-106b) attenuates miR-106b-mediated reduction of angiogenesis. In some embodiments, the antagonist of miR-106b (e.g., antisense oligonucleotide to miR-106b) attenuates miR-106b-mediated reduction of angiogenesis under PAD-relevant conditions. In some embodiments, the antagonist of miR-106b attenuates miR-106b-mediated reduction of angiogenesis by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, the antagonist of miR-106b e.g., antisense oligonucleotide to miR-106b), enhances angiogenesis. In some embodiments, the antagonist of miR-106b enhances angiogenesis by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. Angiogenesis can be measured by any means known in the art. For example, angiogenesis can be measured via assessment of endothelial branching, e.g., via an in vitro angiogenesis assay.

In some embodiments, the antagonist of miR-106b (e.g., the antisense oligonucleotide to miR-106b), attenuates miR-106b-induced reduction of cell proliferation. In some embodiments, the antagonist of miR-106b (e.g., antisense oligonucleotide to miR-106b) attenuates miR-106b-induced reduction of cell proliferation under PAD-relevant conditions. In some embodiments, the antagonist of miR-106b attenuates miR-106b-mediated reduction of cell proliferation by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, the antagonist of miR-106b (e.g., antisense oligonucleotide to miR-106b) enhances angiogenesis. In some embodiments, the antagonist of miR-106b, e.g., the antisense oligonucleotide to miR-106b, enhances cell proliferation. In some embodiments, the antagonist of miR-106b enhances cell proliferation by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, or at least about 300%. The cell proliferation can comprise, e.g., proliferation of endothelial cells. The cell proliferation can comprise, e.g., proliferation of muscle cells, such as, e.g., skeletal muscle cells or cardiac muscle cells. The cell proliferation can be measured in vitro, e.g., in cultured cells (such as, e.g., human umbilical vein endothelial cells (HUVECS) and/or C2C12 cells). The cell proliferation can be measured in vivo.

In some embodiments, the antagonist of miR-106b (e.g., antisense oligonucleotide to miR-106b) attenuates miR-106b-mediated apoptosis. In some embodiments, the antagonist of miR-106b (e.g., antisense oligonucleotide to miR-106b) attenuates miR-106b-mediated apoptosis under PAD-relevant conditions. In some embodiments, the antagonist of miR-106b (e.g., antisense oligonucleotide to miR-106b) attenuates apoptosis induced by hypoxia or ischemia conditions. In some embodiments, the antagonist of miR-106b reduces apoptosis by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. Apoptosis, as well as apoptosis induced by hypoxia or ischemia conditions, can be measured by any means known in the art. The apoptosis can comprise apoptosis of, e.g., endothelial cells and/or muscle cells (e.g., skeletal muscle cells or cardiac muscle cells). The apoptosis can be measured in vitro, e.g., in cultured cells (such as, e.g., HUVECs or C2C12 cells) using a TUNEL assay. The apoptosis can be measured in vivo, e.g., in any animal model of PAD-relevant conditions known in the art. An exemplary model of PAD-relevant conditions is the hind-limb ischemia animal model.

In some embodiments, the antagonist of miR-106b (e.g., antisense oligonucleotide to miR-106b, enhances reperfusion (also referred to herein as perfusion recovery) following ischemia. In some embodiments, the antagonist of miR-106b enhances reperfusion following ischemia by at least about 2.5%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. The ischemia can be ischemia of a limb. Reperfusion following ischemia can be evaluated by any means known in the art. An exemplary model of ischemia includes hind limb ischemia in test rodents, e.g., mice. Reperfusion following hind limb ischemia in test rodents can be evaluated, e.g., by imaging, and/or by measuring capillary density after hind limb ischemia. Exemplary methods for measuring capillary density after hind limb ischemia and for imaging perfusion recovery are disclosed herein. (See, e.g., the Examples within the “Materials and Methods” section).

### Methods of Treatment

For therapeutic applications, miR-106b antagonists are administered to a subject, generally a mammal, generally wherein the mammal is a human, in a pharmaceutically acceptable dosage form such as those known in the art and those discussed above. For example, an miR-106b antagonist may be administered to a human by any administration route, e.g., oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral. In some embodiments, the miR-106b antagonist is administered intramuscularly.

Accordingly, provided herein is a method of treating or preventing a disease, disorder, injury, or condition associated with ischemia in a subject in need thereof, comprising administering an effective amount of an miR-106b antagonist disclosed herein.

In some embodiments, the ischemia is selected from the group consisting of vascular ischemia, muscular ischemia, peripheral arterial disease, ischemia reperfusion injury, ischemia associated with trauma, and brain ischemia, optionally wherein the ischemia is peripheral arterial disease.

In some embodiments, the ischemia is peripheral arterial disease.

In some embodiments, the ischemia is ischemia reperfusion injury.

In some embodiments, the ischemia is brain ischemia. In some embodiments, the brain ischemia is associated with trauma.

In one aspect, the ischemia is vascular ischemia. In one aspect, the vascular ischemia is coronary artery ischemia.

Also provided herein is a method of decreasing expression of, or attenuating ischemia-induced upregulation of, at least one cell cycle pathway gene in an endothelial or muscle cell of a subject in need thereof, comprising administering an effective amount of an miR-106b antagonist disclosed herein. Exemplary cell cycle pathway genes are disclosed herein.

Also provided herein is a method of enhancing perfusion recovery in a subject in need thereof, comprising administering an effective amount of an miR-106b antagonist disclosed herein.

Also provided herein is a method of enhancing angiogenic response to ischemia in a subject in need thereof, comprising administering an effective amount of an miR-106b antagonist disclosed herein.

Also provided herein is a method of stimulating cell proliferation in a subject following an ischemic event in the subject, comprising administering an effective amount of an miR-106b antagonist disclosed herein. In some embodiments, the cell proliferation is proliferation of endothelial and/or muscle cells.

Also provided herein is a method of increasing capillary density in a subject in need thereof, comprising administering an effective amount of an miR-106b antagonist disclosed herein.

Also provided herein is a method of inhibiting apoptosis of one or more cells in a subject in need thereof, comprising administering an effective amount of an miR-106b antagonist disclosed herein. In some embodiments, the apoptosis comprises hypoxia-induced apoptosis, e.g., apoptosis induced by ischemia-induced hypoxia. In some embodiments, the one or more cells are endothelial cells and/or muscle cells.

### Pharmaceutical Compositions

In some embodiments, provided are pharmaceutical compositions comprising an miR-106b antagonist, wherein the pharmaceutical composition further comprises another miRNA molecule and wherein the miR-106b antagonist is duplexed with the other miRNA molecule. In some embodiments of such pharmaceutical composition, the miR-106b antagonist is an antisense oligonucleotide of hsa-miR-106b-5p, and is duplexed with miR-93. In some embodiments of such pharmaceutical compositions, the pharmaceutical composition comprises a duplex of (1) an antisense oligonucleotide of hsa-miR-106b-5p, the antisense oligonucleotide comprising the sequence AUCUGCACUGUCAGCACUUUA (SEQ ID NO: 6) and (2) a sequence comprising hsa-miR-93-5p sequence AAAGUGCUGUUCGUGCAGGUAG (SEQ ID NO: 3). In some embodiments of such pharmaceutical compositions, the pharmaceutical composition comprises a duplex of (1) an antisense oligonucleotide of hsa-miR-106b-5p, the antisense oligonucleotide comprising the sequence AUCUGCACUGUCAGCACUUUA (SEQ ID NO: 6) and (2) a sequence comprising hsa-miR-93-5p sequence CAAAGUGCUGUUCGUGCAGGUAG (SEQ ID NO: 4).

The antagonists (e.g., antisense oligonucleotides) described herein can be formulated in pharmaceutical compositions. These compositions can comprise, in addition to one or more of the antagonists disclosed herein, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration (e.g. oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral routes of administration).

Pharmaceutical compositions for oral administration can be, e.g., in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

In some embodiments, the pharmaceutical composition is formulated for intramuscular administration.

Administration of the one or more antagonists disclosed herein is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical professionals, and typically takes account of the disorder to be treated, the condition of the individual patient, the site of delivery, the method of administration, avoidance of side effects, and other factors known to practitioners. Examples of the techniques and protocols mentioned above can be found, e.g., in Remington's Pharmaceutical Sciences, 16th edition, Osol, A. (ed), 1980.

A composition can be administered alone or in combination with other treatments, either simultaneously or sequentially depending upon the condition to be treated.

Pharmaceutical compositions that are useful in the methods of the disclosure may be prepared, packaged, or sold in formulations suitable for the desired route of administration (e.g., oral, rectal, vaginal, parenteral, topical, pulmonary, intranasal, buccal, ophthalmic, intrathecal or another route of administration). Other contemplated formulations include projected nanoparticles, liposomal preparations, resealed erythrocytes containing the active ingredient, and immunologically-based formulations.

A pharmaceutical composition of the present disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the present disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the present disclosure may further comprise one or more additional pharmaceutically active agents. Particularly contemplated additional agents include anti-emetics and scavengers such as cyanide and cyanate scavengers.

Controlled- or sustained-release formulations of a pharmaceutical composition of the present disclosure may be made using conventional technology. A formulation of a pharmaceutical composition of the present disclosure suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the present disclosure which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Examples of aqueous vehicles include, for example, water and isotonic saline. Examples of oily vehicles include, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients, examples of which include, but are not limited to, suspending agents, dispersing or wetting agents, emulsifying agents, demulcents, preservatives, buffers, salts, flavorings, coloring agents, and sweetening agents. Oily suspensions may further comprise a thickening agent. Examples of known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Examples of known dispersing or wetting agents include, but are not limited to, naturally occurring phosphatides such as lecithin and condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, or polyoxyethylene sorbitan monooleate, respectively).

Examples of known emulsifying agents include, but are not limited to, lecithin and acacia. Examples of known preservatives include, but are not limited to, methyl, ethyl, or n-propyl para hydroxybenzoates, ascorbic acid, and sorbic acid. Examples of known sweetening agents include, but are not limited to, glycerol, propylene glycol, sorbitol, sucrose, and saccharin. Examples of known thickening agents for oily suspensions include, but are not limited to, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the present disclosure may comprise each of the components described with regard to liquid suspensions, it being understood that suspending agents will not necessarily aid dissolution of the active ingredient in the solvent. Examples of aqueous solvents include, but are not limited to, water and isotonic saline. Examples of oily solvents include, but are not limited to, almond oil, oily esters, ethyl alcohol, vegetable oils such as arachis, olive, sesame, or coconut oil, fractionated vegetable oils, and mineral oils such as liquid paraffin.

A composition of the present disclosure may comprise additional ingredients. As used herein, examples of “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Examples of other “additional ingredients” which may be included in the pharmaceutical compositions of the present disclosure are known in the art and are described, for example in Genaro, ed., 1985, Remington's Pharmaceutical Sciences, Mack Publishing Co., Easton, Pa., which is incorporated herein by reference.

The pharmaceutical composition may be administered to an animal as frequently as several times daily, or it may be administered less frequently, such as once a day, once a week, once every two weeks, once a month, or even less frequently, such as once every several months or even once a year or less. The frequency of the dose is typically determined by the skilled artisan and may depend upon any number of factors, such as, but not limited to, the type and severity of the condition or disease being treated, the type and age of the animal, etc.

### Combination Therapy

Compositions and methods of treatment can comprise co-administration of one or more miR-106b antagonists disclosed herein with one or more additional agents. Examples of additional agents include, but are not limited to cytotoxic agents, anti-angiogenic agents, pro-apoptotic agents, antibiotics, hormones, hormone antagonists, chemokines, drugs, prodrugs, toxins, and enzymes.

Further examples of additional agents include, but are not limited to (a) antimicrobials, (b) steroids (e.g., hydrocortisone, triamcinolone); (c) pain medications (e.g., aspirin, an NSAID, and a local anesthetic); (d) anti-inflammatory agents; (e) growth factors; (f) cytokines; (g) hormones; (h) other agents for treatment of peripheral arterial disease, and (i) combinations thereof.

In some embodiments, the one or more additional agents comprises an antisense oligonucleotide to another miRNA molecule. In some embodiments, the other miRNA molecule is miR-93. In some embodiments, the miR-93 comprises the sequence AAAGUGCUGUUCGUGCAGGUAG (has-miR-93-3p; SEQ ID NO: 3). In some embodiments, the miR-93 comprises the sequence CAAAGUGCUGUUCGUGCAGGUAG (hsa-miR-93-5p; SEQ ID NO: 4). In some embodiments, the combination therapy with the other miRNA molecule comprises administering a pharmaceutical composition comprising the miR-106b antagonist to a subject, e.g., a human subject in need thereof, wherein the pharmaceutical composition comprises the other miRNA molecule. In some such embodiments, the miR-106b antagonist is duplexed with the other miRNA molecule. Also provided herein is a pharmaceutical composition comprising the miR-106b antagonist, wherein the pharmaceutical composition further comprises the other miRNA molecule and wherein the miR-106b antagonist is duplexed with the other miRNA molecule. In some embodiments of such pharmaceutical composition, the miR-106b antagonist is an antisense oligonucleotide of hsa-miR-106b-5p, and is duplexed with miR-93. In preferred embodiments of such pharmaceutical composition, the pharmaceutical composition comprises a duplex of an antisense oligonucleotide of hsa-miR-106b-5p (e.g., an antisense oligonucleotide comprising the sequence of AUCUGCACUGUCAGCACUUUA (SEQ ID NO: 6)) and a miR-93 sequence comprising AAAGUGCUGUUCGUGCAGGUAG (SEQ ID NO: 3). In yet other embodiments of such pharmaceutical compositions, the pharmaceutical composition comprises a duplex of the an antisense oligonucleotide of hsa-miR-106b-5p (e.g., an antisense oligonucleotide comprising the sequence AUCUGCACUGUCAGCACUUUA (SEQ ID NO: 6)) and a miR-93 sequence comprising

(SEQ ID NO: 4)
CAAAGUGCUGUUCGUGCAGGUAG.

In some embodiments, methods also comprise use of a therapeutic molecule including, without limitation, any pharmaceutical or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfiram and disulfiram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatoires, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitors. All substances listed by the U.S. Pharmacopeia may also be included.

A list of the types of drugs, and specific drugs within categories whose uses may be encompassed within the present disclosure is provided below; this list is intended to provide non-limiting examples.

Antimicrobial agents include: silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), Neosporin (i.e., Bacitracin, Polymyxin B, and Neomycin), Polysporin (i.e., Bacitracin and Polymyxin B). Additional antimicrobials include topical antimicrobials (i.e., antiseptics), examples of which include silver salts, iodine, benzalkonium chloride, alcohol, hydrogen peroxide, and chlorhexidine.

Analgesic: Acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin.

Antihypertensive: Aflyzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Malate; Ditekiren; Doxazosin Mesylate; Ecadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride; Pazoxide; Pelanserin Hydrochloride; Perindopril Erbumine; Phenoxybenzamine Hydrochloride; Pinacidil; Pivopril; Polythiazide; Prazosin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Ticrynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine.

Anti-inflammatory: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

In some embodiments, an effective amount of at least one growth factor, cytokine, hormone, or extracellular matrix compound or protein useful for enhancing wound healing is administered. In one aspect, a combination of these agents is used. In one aspect, examples of growth factors useful in the practice of the present disclosure include, but are not limited to, EGF, PDGF, GCSF, IL6, IL8, IL10, MCP1, MCP2, Tissue Factor, FGFb, KGF, VEGF, PLGF, MMP1, MMP9, TIMP1, TIMP2, TGF.beta., and HGF. One of ordinary skill in the art will appreciate that the choice of growth factor, cytokine, hormone, or extracellular matrix protein used will vary depending on criteria such as the type of injury, disease, or disorder being treated, the age, health, sex, and weight of the subject, etc. In one aspect, the growth factors, cytokines, hormones, and extracellular matrix compounds and proteins are human.

Examples of proteins and other biologically active compounds that can be incorporated into, or included as an additive within, a composition comprising compounds of the present disclosure include, but are not limited to, collagen (including cross-linked collagen), fibronectin, laminin, elastin (including cross-linked elastin), osteopontin, osteonectin, bone sialoproteins (Bsp), alpha-2HS-glycoproteins, bone Gla-protein (Bgp), matrix Gla-protein, bone phosphoglycoprotein, bone phosphoprotein, bone proteoglycan, protolipids, bone morphogenetic protein, cartilage induction factor, skeletal growth factor, enzymes, or combinations and biologically active fragments thereof. Adjuvants that diminish an immune response can also be used in conjunction with the composite of the subject disclosure.

Examples of other molecules that may be useful as compounds or substances in the present disclosure include, but are not limited to, growth hormones, leptin, leukemia inhibitory factor (LIF), tumor necrosis factor alpha and beta, endostatin, angiostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18. Embodiments involving amino acids, peptides, polypeptides, and proteins may include any type of such molecules of any size and complexity as well as combinations of such molecules.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Materials and Methods

Molecules and duplexes of interest: One or more of the following molecules or duplexes may be tested in the following examples:
(1) an antagonist of miR-106b, e.g., an antisense oligonucleotide (e.g., antisense RNA oligonucleotide. For example,
   i. “Molecule 1:” an RNA oligonucleotide comprising or having the sequence AUCUGCACUGUCAGCACUUUA (SEQ ID NO: 6)
(2) a miR-93 molecule. For example,
   i. “Molecule 2:” an RNA oligonucleotide comprising or having the sequence AAAGUGCUGUUCGUGCAGGUAG (SEQ ID NO: 3)
   ii. “Molecule 3:” an RNA oligonucleotide comprising or having the sequence CAAAGUGCUGUUCGUGCAGGUAG (SEQ ID NO: 4)
(3) a duplex of (a) an antisense oligonucletotide of hsa-miR-106b and (b) another nucleic acid molecule, e.g., the other nucleic acid molecule having a miR-93 sequence. For example,
   i. “Duplex 1:” Molecule 1 duplexed together with an RNA oligonucleotide comprising or having the sequence

(SEQ ID NO: 3)
AAAGUGCUGUUCGUGCAGGUAG.

ii. “Duplex 2:” Molecule 1 duplexed together with an RNA oligonucleotide having the sequence

(SEQ ID NO: 4)
CAAAGUGCUGUUCGUGCAGGUAG.

While any duplexes such as Duplex 1 and Duplex 2 may be provided to a subject in duplex form, in some embodiments, the duplex may disassociate once inside the subject's body into separate strands. For example, Duplex 1 and 2, may, in some embodiments, separate into Molecule 1 and the other RNA oligonucleotide inside a subject's body.

In some embodiments, administration or delivery of Molecule 1, Duplex 1, and/or Duplex 2 lead to knockdown of miR-106b. In some embodiments, administration or delivery of Duplex 1 and/or Duplex lead to overexpression of miR-93.

In some Examples, results from experiments using Molecule 1, Molecule 2, and/or Molecule 3 are compared against results from experiments using Duplex 1 or Duplex 2. In some Examples, superior results may be observed in experiments using Duplex 1 or Duplex 2 as compared to experiments using Molecule 1, 2, or 3.

Murine Model of Hind-limb Ischemia and Monitoring of Perfusion Recovery: Animal studies are approved by the Institutional Animal Care Committee and conform to the Guide for the Care and Use of Laboratory Animals published by the US National Institute of Health. After inducing anesthesia (ketamine 90 mg/kg and xylazine 10 mg/kg), unilateral femoral artery ligation and excision is performed on 8-12 weeks old male C57B1/6J or BALB/cJ mice. Perfusion recovery is measured using laser Doppler imaging (Perimed, Inc.) on days 0, 3, 7, 14, and 21 post-surgery. Perfusion in the ischemic limb is normalized to that in the non-ischemic limb for each mouse.

Micro-RNA Micro-array: Gastrocnemius muscles are collected from C57B1/6J and BALB/cJ mice (n=3/group) at day 3 post-surgery. RNA is isolated using TRIzol™ total transcriptome isolation protocol and Pure Link RNA Mini kits following manufacturer's instructions (Life Technologies, Carlsbad, Calif.). RNA is aliquoted into 50 μg amounts, and arrays are done using Illumina micro-RNA chips containing 380 mouse micro-RNAs (V2 Micro-RNA Expression Profiling Kit; miRbase 9.1). Differentially expressed micro-RNAs are sorted based on regulation by a combination of ischemia and strain, or regulation by ischemia or strain alone.

mRNA Micro-array: For mouse mRNA arrays between ischemic and non-ischemic tissue, gastrocnemius muscle from BALB/cJ mice (n=3/group) are harvested at day 3-post-hind limb ischemia (HLI). Total RNA is extracted using TRIzol™ total transcriptome isolation protocol. After quality control, RNAs are aliquoted into 50 μg aliquots and arrays are done using Gene Chip mouse genome 43-2.0. For HUVEC mRNA arrays, RNA from HUVECs are isolated after HUVECs are transfected with scrambled sequences or with antimiR-106 and incubated for 24-hours under conditions of hypoxia and serum starvation (total of 48 hrs post-transfection). Arrays are done using Illumina Human 6 V 1 platform. Non-normalized data are obtained using Illumina's Genome Studio. Quality control, pre-processing and quantile normalization is done using R and the beadarray package. Analysis for gene set enrichment is done using GSEA V 2.0 from Broad Institute, using 100 permutations and FDR cutoff of <0.25.

In vivo knockdown of miR-106b: AntagomiR-106b and scrambled sequences are synthesized. Oligo sequences are as follows: AntagomiR-106b: 5′-AUCUGCACUGUCAGCACUUUA-3′ (SEQ ID NO: 6). Scramble: 5′-AAGGCAAGCUGACCCUGAAGUU-3′ (SEQ ID NO: 10). Oligos are dissolved in PBS and injected retro-orbitally at a dose of 8 mg/kg body weight. Injections are given 30-minutes prior to, and at days 7 and 14 post-HLI. For detection of the efficiency of in-vivo knockdown, a separate group of mice are euthanized at days 0, 3, and 7, and miR-106b expression is quantitated using real-time qPCR.

Cell Culture: Pooled HUVECs are purchased (Cell Applications Inc, San Diego, Calif.), and grown in standard endothelial cell growth medium with 10% FBS (Cell Applications Inc, San Diego, Calif.). C2C12 cells are cultured on DMEM with 10% FBS. For in-vitro transfection studies, a reverse transfection protocol using neofx transfection agent (Ambion, Austin, Tex.) is used. AntimiR-106b, miRNA inhibitor negative control (Cat. #44640760), premiR-106b or miRNA mimic negative control (Cat. #4464058) are purchased from Ambion, Austin, Tex. Initial dose response experiments are done on HUVECs and C2C12 cells to determine the dose and time course for efficient knockdown of miR-106b. Based on these experiments, a dose of 15 nM of antimiR-106b or premiR-106b and their respective controls are used to knockdown or over-express miR-106b in HUVECs. For C2C12 cells, a dose of 120 nM is used for antimiR-106b or premiR-106b and their respective controls. Cell proliferation, tube formation and apoptosis assays are done 48 hours after transfection.

Cellular Apoptosis: Cells are plated in a 96-well plate at a density of 1×10⁴ cells/well for HUVECs and for C2C12 cells, cells are plated at a density of 0.5×10⁴ (for premiR-106b vs. Scramble) or 1×10⁵ (Scramble vs. AntimiR-106b). After 24-hours of transfection, miR-106b modulated cells are exposed to hypoxia (2% oxygen, BioSpherix, Lacona, N.Y.) and serum starvation (HSS) to simulate ischemia in vitro. HUVECs are exposed to 48-hours of HSS, while C2C12 cells are exposed to 3-hours of HSS. A shorter time course of exposure to HSS for C2C12 cells are selected based on preliminary experiments that showed that C2C12 cells show significant cell death with longer duration of HSS. At the end of incubation, apoptosis in cells is determined using a TUNEL assay (TiterTACS, Trevigen Gaithersburg, Md.). TACS nuclease treated wells are used as positive control, while wells without addition of TdTs are used as negative controls. Each experiment is repeated at least three times.

Cell Proliferation: Cells are plated in a 96-well plate at a plating density of 5×10³ cells/well for HUVECs and at a density of 0.5×10³ (for premiR-106b vs. Scramble) or 1×10³ (Scramble vs. AntimiR-106b) for C2C12 cells. Cell proliferation is assessed 48 and 72 hours after plating using tetrazolium dye incorporation (BioVision, Milpitas, Calif.), and by doing manual cell counts after trypan blue staining to exclude dead cells. Experiments are repeated three times.

In vitro Angiogenesis Assay: After 48-hours of transfection, miR-106b modulated HUVECs are plated on matrigel to assess tube formation. PremiR-106b or Scramble transfected cells are plated on growth factor reduced matrigel (Cat #356231, BD Biosciences, Bedford, Mass.) at a cell density of 30,000 cells/well in a 48-well plate, and cells are cultured under conditions of 0% or 5% low serum growth medium (Life Technologies, NY). Similarly, antimiR-106b or scramble transfected cells are plated on growth factor enriched matrigel (Cat. #356234, BD Biosciences, Bedford, Mass.), and grown under conditions of 0% or 5% mixture of endothelial cell growth factors (EGM CC-3124, Lonza, Allendale, NJ). Endothelial cell tube formation is assessed 6 hours after plating. Each condition is done in triplicates. Four representative pictures are taken from each well under 100× magnification, and total complete tube numbers are counted and expressed as tube numbers per square mm.

Capillary Density: For assessment of capillary density, 21 days post-HLI, ischemic gastrocnemius muscles from premiR-106b and Scramble treated BALB/cJ mice are flash frozen in OCT compound and sectioned at 7 μm thickness. Sections are first blocked with 5% normal goat serum, and then incubated with rat anti-CD31 antibody (1:25, BD Biosciences cat #550274) at 4° C. overnight. Sections are then washed with PBS and probed with Alexa-555 conjugated goat anti-rat IgG at 1:25 dilution for 1 hour at room temperature. Sections are washed with PBS and mounted with Vectashield mounting medium (Vector Lab, Burlingame, Calif.). Secondary antibody only without primary antibody is used as negative control to assess non-specific binding. Three representative pictures from each section are taken under 400× magnification, using Olympus BX51 high-magnification microscope. Total number of CD31 positive spots/field and total number of muscle fiber/field are counted, and capillaries expressed as CD31 positive spots/muscle fiber.

### Example 1: Knockdown of miR-106b and/or Overexpression of miR-93 in Cultured Cells

miR-106b is knocked down and/or miR-93 is overexpressed in HUVECs and C2C12 cells. Scramble antimir sequences are used as controls. HUVECs and C2C12 cells are cultured and treated with antimiR-106b (e.g., Molecule 1), a miR-93 molecule (e.g., Molecule 2 or Molecule 3), a duplex (e.g., Duplex 1 or Duplex 2) or scramble antimiR-106b sequences as described above. miR-106b levels and/or miR-93 after transfection are assessed. A reduction of at least 50% miR-106b would indicate significant reduction; an increase of at least 50% miR-93 would indicate a significant increase.

Following HSS, effects of knockdown of miR-106b and/or effects of miR-93 overexpression on caspase-9, a marker of apoptosis in both HUVECs and C2C12 cells, are assessed. Attentuation of caspase-9 after transfection would indicate that miR-106b knockdown and/or miR-93 overexpression mediates cell survival in response to HSS in both endothelial and skeletal muscle cells.

Results from cells transfected with Duplex 1 or Duplex 2 are compared to those in cells transfected with Molecule 1, Molecule 2, or Molecule 3.

### Example 2: Effects of Molecules and/or Duplexes on Cell Proliferation

The angiogenic response to ischemia includes endothelial cell survival, proliferation, and migration. The effects of Molecule 1, Molecule 2, Molecule 3, Duplex 1, and/or Duplex 2 on cell proliferation (e.g., in endothelial and C2C12 cells) is investigated according to methods disclosed herein. (See, e.g., “Materials and Methods” above.) Results from cells transfected with Duplex 1 or Duplex 2 are compared to those from cells transfected with Molecule 1, Molecule 2, or Molecule 3.

### Example 3: Impact of Molecules and/or Duplexes on Perfusion Recovery Following Hind Limb Ischemia

Mice are treated with systemically delivered Molecule 1, Molecule 2, Molecule 3, Duplex 1, and/or Duplex 2 to test whether mir-106b antagonism and/or miR-93 overexpression modulate the response to HLI. Scramble treated mice are used as controls. A single intravenous injection of Molecule 1, Molecule 2, Molecule 3, Duplex 1, and/or Duplex 2 is given 30-minutes prior to surgery.

Effects on miR-106b expression (e.g., knockdown of expression) and/or of miR-93 expression (e.g., overexpression) are assessed starting at day-1 and at later timepoints, e.g., at 7-days post-injection. Molecule 1, Molecule 2, Molecule 3, Duplex 1, and/or Duplex 2 or scramble sequences are injected on day 0, 7, and 14 of HLI, and perfusion recovery is monitored using Doppler imaging. Improved perfusion recovery in antagomiR-106b (e.g., Molecule 1)-treated mice compared to scramble treated mice after HLI would indicate that inhibiting miR-106b is effective for enhancing perfusion recovery.

Results from Duplex 1 or Duplex 2-treated mice are compared to those from mice treated with Molecule 1, Molecule 2, or Molecule 3.

### Example 4: Effects of Molecules and Duplexes on Multiple Genes In Vivo

p21, p53, and E2F-1 are up-regulated in the ischemic vs. non-ischemic muscle. BALB/cJ mice are treated with Molecule 1, Molecule 2, Molecule 3, Duplex 1, and/or Duplex 2, and p21, p53, and E2F-1 expression is assessed compared to that in scramble treated mice. Downregulation of p21, p53, and E2F-1 in mice treated with Molecule 1, Molecule 2, Molecule 3, Duplex 1, or Duplex 2 (but not in scramble treated mice) would indicate that miR-106b knockdown and/or miR-93 regulates these genes in vivo.

Results from Duplex 1 or Duplex 2-treated mice are compared to those from mice treated with Molecule 1, Molecule 2, or Molecule 3.

### Example 5: Effects of Molecules and Duplexes on miR-106b-Mediated Inhibition of Endothelial Branching

The effect of miR-106b knockdown and/or miR-93 overexpression on endothelial cell tube formation in matrigel models is assessed according to an in vitro angiogenesis assay described herein. miR-106b reduces endothelial cell tube formation by about 50%.

In the present example, the effect of Molecule 1, Molecule 2, Molecule 3, Duplex 1, and/or Duplex 2 on endothelial cell tube formation is assessed. Reversal of the effect of miR-106b on endothelial cell tube formation would indicate a pro-angiogenic role of miR-106b knockdown and/or miR-93 expression in vitro.

Results from experiments with Duplex 1 or Duplex 2 are compared to those from experiments with Molecule 1, Molecule 2, or Molecule 3.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### NUMBERED EMBODIMENTS

Embodiment 1: A method of treating or preventing a disease, disorder, injury, or condition associated with ischemia, said method comprising administering to a subject in need thereof a pharmaceutical composition comprising an effective amount of an antagonist of miRNA expression, levels, or activity, a pharmaceutically-acceptable carrier, wherein said miRNA is miR-106b, thereby treating said disease, disorder, or condition associated with ischemia.

Embodiment 2: The method of embodiment 1, wherein the miR-106b is human miR-106b.

Embodiment 3: The method of embodiment 2, wherein the human miR-106b is human miR-106b-5p comprising the sequence UAAAGUGCUGACAGUGCAGAU (SEQ ID NO: 1).

Embodiment 4: The method of embodiment 2, wherein the human miR-106b is human miR-106b-3p comprising the sequence CCGCACUGUGGGUACUUGCUGC (SEQ ID NO: 2).

Embodiment 5: The method of any one of the preceding embodiments, wherein the antagonist is an antisense oligonucleotide comprising a sequence that is fully or partially complementary to a portion of mature miR-106b such that the antisense oligonucleotide binds to miR-106b.

Embodiment 6: The method of any one of the preceding embodiments, wherein the antisense oligonucleotide comprises DNA.

Embodiment 7: The method of any one of the preceding embodiments, wherein the antisense oligonucleotide comprises RNA.

Embodiment 8: The method of any one of the preceding embodiments, wherein the antisense oligonucleotide is an antagomir of miR-106b.

Embodiment 9: The method of any one of the preceding embodiments, wherein the antisense oligonucleotide comprises one or more nucleotide analogs.

Embodiment 10: The method of embodiment 9, wherein the one or more nucleotide analogs comprises LNA.

Embodiment 11: The method of any one of the preceding embodiments, wherein the antisense oligonucleotide is capable of forming a duplex with a mature miR-106b molecule, the duplex having a Ti of at least about 60° C.

Embodiment 12: The method of any one of the preceding embodiments, wherein the antisense oligonucleotide is capable of forming a duplex with another single stranded RNA nucleic acid molecule, the duplex having a Tₘ of less than about 60° C., less than about 50° C., less than about 45° C., less than about 40° C., less than about 37° C., less than about 35° C., less than about 30° C., or less than about 25° C.

Embodiment 13: The method of any one of the preceding embodiments, wherein the antagonist decreases miR-106b expression, levels, or activity.

Embodiment 14: The method of any one the preceding embodiments, further comprising administering to the subject an additional therapeutic agent.

Embodiment 15: The method of embodiment 14, wherein said additional therapeutic agent comprises an anti-ischemia agent.

Embodiment 16: The method of any one of the preceding embodiments, wherein the antagonist is encoded by an isolated nucleic acid or vector comprising the isolated nucleic acid.

Embodiment 17: The method of embodiment 16, wherein said vector is an expression vector selected from an miRNA expression vector or AAV expression vector.

Embodiment 18: The method of embodiment 17, wherein said expression vector is an miRNA expression vector.

Embodiment 19: The method of embodiment 15, wherein said isolated nucleic acid is operably-linked to a cell-specific promoter.

Embodiment 20: The method of any one of the preceding embodiments, wherein the antagonist is encapsulated within a lipid vehicle.

Embodiment 21: The method of any one of the preceding embodiments, wherein the effective amount is effective to decrease expression of, or attenuate ischemia-induced upregulation of, at least one cell cycle pathway gene in an endothelial or muscle cell of the subject.

Embodiment 22: The method of embodiment 21, wherein said cell cycle pathway genes are selected from the group consisting of E2F-1 and p53.

Embodiment 23: The method of any one of embodiments 16-22, wherein said expression is in skeletal muscle cells.

Embodiment 24: The method of any one of the preceding embodiments, wherein the effective amount is effective to enhance perfusion recovery in the subject.

Embodiment 25: The method of any one of the preceding embodiments, wherein the effective amount is effective to enhance angiogenic response to ischemia in the subject. Embodiment 26: The method of any one of the preceding embodiments, wherein the effective amount is effective to stimulate cell proliferation.

Embodiment 27: The method of embodiment 26, wherein the cell proliferation comprises proliferation of endothelial cells or muscle cells.

Embodiment 28: The method of any one of the preceding embodiments, wherein the effective amount is effective to increase capillary density in the subject.

Embodiment 29: The method of any one of the preceding embodiments, wherein the effective amount is effective to inhibit apoptosis of one or more cells in the subject.

Embodiment 30: The method of embodiment 29, wherein said apoptosis is hypoxia-induced apoptosis.

Embodiment 31: The method of any one of the preceding embodiments, wherein said administration is by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

Embodiment 32: The method of embodiment 31, wherein said administration is intramuscular.

Embodiment 33: The method of any one of the preceding embodiments, wherein said subject is a human.

Embodiment 34: The method of any one of the preceding embodiments, wherein said ischemia is selected from the group consisting of vascular ischemia, muscular ischemia, peripheral arterial disease, ischemia reperfusion injury, ischemia associated with trauma, and brain ischemia, optionally wherein the ischemia is peripheral arterial disease.

Embodiment 35: A pharmaceutical composition, comprising an effective amount of an antagonist of miRNA expression, levels, or activity, a pharmaceutically-acceptable carrier, wherein said miRNA is miR-106b, and a pharmaceutically acceptable carrier.

Embodiment 36: The pharmaceutical composition of embodiment 35, wherein the miR-106b is human miR-106b.

Embodiment 37: The pharmaceutical composition of embodiment 36, wherein the human miR-106b is human miR-106b-5p comprising the sequence

(SEQ ID NO: 1)
UAAAGUGCUGACAGUGCAGAU.

Embodiment 38: The pharmaceutical composition of embodiment 36, wherein the human miR-106b is human miR-106b-3p comprising the sequence

(SEQ ID NO: 2)
CCGCACUGUGGGUACUUGCUGC.

Embodiment 39: The pharmaceutical composition of any one of embodiments 35-38, wherein the antagonist is an antisense oligonucleotide comprising a sequence that is fully or partially complementary to a portion of mature miR-106b such that the antisense oligonucleotide binds to miR-106b.

Embodiment 40: The pharmaceutical composition of any one of embodiments 35-39, wherein the antisense oligonucleotide comprises DNA.

Embodiment 41: The pharmaceutical composition of any one of embodiments 35-40, wherein the antisense oligonucleotide comprises RNA.

Embodiment 42: The pharmaceutical composition of any one of embodiments 35-41, wherein the antisense oligonucleotide is an antagomir of miR-106b.

Embodiment 43: The pharmaceutical composition of any one of embodiments 35-42, wherein the antisense oligonucleotide comprises one or more nucleotide analogs.

Embodiment 44: The pharmaceutical composition of embodiment 43, wherein the one or more nucleotide analogs comprises LNA.

Embodiment 45: The pharmaceutical composition of any one of embodiments 35-44, wherein the antisense oligonucleotide is capable of forming a duplex with a mature miR-106b molecule, the duplex having a Tₘ of at least about 60° C.

Embodiment 46: The pharmaceutical composition of any one of embodiments 35-45, wherein the antisense oligonucleotide is capable of forming a duplex with another single stranded RNA nucleic acid molecule, the duplex having a Tₘ of less than about 60° C., less than about 50° C., less than about 45° C., less than about 40° C., less than about 37° C., less than about 35° C., less than about 30° C., or less than about 25° C.

Embodiment 47: The pharmaceutical composition of any one of embodiments 35-46, wherein the antagonist decreases miR-106b expression, levels, or activity.

Embodiment 48: The pharmaceutical composition of any one of embodiments 35-47, further comprising an additional therapeutic agent.

Embodiment 49: The pharmaceutical composition of embodiment 48, wherein said additional therapeutic agent comprises an anti-ischemia agent.

Embodiment 50: The pharmaceutical composition of any one of embodiments 35-49, wherein the antagonist is encoded by an isolated nucleic acid, or vector comprising the isolated nucleic acid.

Embodiment 51: The pharmaceutical composition of embodiment 50, wherein said vector is an expression vector selected from an miRNA expression vector or AAV expression vector.

Embodiment 52: The pharmaceutical composition of embodiment 51, wherein said expression vector is an miRNA expression vector.

Embodiment 53: The pharmaceutical composition of embodiment 50, wherein said isolated nucleic acid is operably-linked to a cell-specific promoter.

Embodiment 54: The pharmaceutical composition of any one of embodiments 35-53, wherein the antagonist is encapsulated within a lipid vehicle.

Embodiment 55: The pharmaceutical composition of any one of embodiments 35-54, wherein the effective amount is effective to decrease expression of at least one cell cycle pathway gene in an endothelial or muscle cell of the subject.

Embodiment 56: The pharmaceutical composition of embodiment 55, wherein said cell cycle pathway genes are selected from the group consisting of E2F-1 and p53.

Embodiment 57: The pharmaceutical composition of embodiment 55 or 56, wherein said expression is in skeletal muscle cells.

Embodiment 58: The pharmaceutical composition of any one of embodiments 35-57, wherein the effective amount is effective to enhance perfusion recovery in the subject.

Embodiment 59: The pharmaceutical composition of any one of embodiments 35-58, wherein the effective amount is effective to enhance angiogenic response to ischemia in the subject.

Embodiment 60: The pharmaceutical composition of any one of embodiments 35-59, wherein the effective amount is effective to stimulate cell proliferation.

Embodiment 61: The pharmaceutical composition of embodiment 60, wherein the cell proliferation comprises proliferation of endothelial cells or muscle cells.

Embodiment 62: The pharmaceutical composition of any one of embodiments 35-61, wherein the effective amount is effective to increase capillary density in the subject.

Embodiment 63: The pharmaceutical composition of any one of embodiments 35-62, wherein the effective amount is effective to inhibit apoptosis of one or more cells in the subject.

Embodiment 64: The pharmaceutical composition of embodiment 63, wherein said apoptosis is hypoxia-induced apoptosis.

Embodiment 65: The pharmaceutical composition of any one of embodiments 35-64, formulated for administration by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

Embodiment 66: The pharmaceutical composition of embodiment 65, wherein said administration is intramuscular.

Embodiment 67: A kit, comprising a pharmaceutical composition of any one of embodiments 35-66, and instructions for use in treating or preventing a disease, disorder, injury, or condition associated with skeletal muscle ischemia in a subject in need thereof, wherein said disease, disorder, injury, or condition is peripheral arterial disease.

Embodiment 68: An isolated nucleic acid comprising an antisense oligonucleotide of miR-106b, wherein the antisense oligonucleotide is an miR-106b antagomir.

Embodiment 69: The isolated nucleic acid of embodiment 68, wherein the miR-106b is human miR-106b.

Embodiment 70: The isolated nucleic acid of embodiment 69, wherein the human miR-106b is human miR-106b-5p comprising the sequence UAAAGUGCUGACAGUGCAGAU (SEQ ID NO: 1).

Embodiment 71: The isolated nucleic acid of embodiment 69, wherein the human miR-106b is human miR-106b-3p comprising the sequence CCGCACUGUGGGUACUUGCUGC (SEQ ID NO: 2).

Embodiment 72: The isolated nucleic acid of any one of embodiments 68-71, wherein the antagonist is an antisense oligonucleotide comprising a sequence that is fully or partially complementary to a portion of mature miR-106b such that the antisense oligonucleotide binds to miR-106b.

Embodiment 73: The isolated nucleic acid of any one of embodiments 68-72, wherein the antisense oligonucleotide comprises DNA.

Embodiment 74: The isolated nucleic acid of any one of embodiments 68-73, wherein the antisense oligonucleotide comprises RNA.

Embodiment 75: The isolated nucleic acid of any one of embodiments 68-74, wherein the antisense oligonucleotide comprises one or more nucleotide analogs.

Embodiment 76: The isolated nucleic acid of embodiments 75, wherein the one or more nucleotide analogs comprises LNA.

Embodiment 77: The isolated nucleic acid of any one of embodiments 68-76, wherein the one or more nucleotide analogs comprises LNA.

Embodiment 78: The isolated nucleic acid of any one of embodiments 68-77, wherein the antisense oligonucleotide is capable of forming a duplex with another single stranded RNA nucleic acid molecule, the duplex having a Tₘ of less than about 60° C., less than about 50° C., less than about 45° C., less than about 40° C., less than about 37° C., less than about 35° C., less than about 30° C., or less than about 25° C.

Embodiment 79: The isolated nucleic acid of any one of embodiments 68-78, wherein the antagonist decreases miR-106b expression, levels, or activity.

1. A nucleic acid duplex comprising:
(a) a miR-93 nucleic acid molecule; and
(b) an antagonist of miR-106b.

2. The nucleic acid duplex of claim 1, wherein the duplex is an RNA:RNA duplex.

3. The nucleic acid duplex of claim 1 or 2, wherein the antagonist of miR-106b is an antisense oligonucleotide that is fully or partially complementary to at least a portion of miR-106b.

4. The nucleic acid duplex of claim 1, 2 or 3, wherein the duplex comprises a miR-93 RNA comprising the sequence AAAGUGCUGUUCGUGCAGGUAG (SEQ ID NO: 3) or a miR-93 RNA comprising the sequence CAAAGUGCUGUUCGUGCAGGUAG (SEQ ID NO: 4).

5. The nucleic acid duplex of any one of claims 1-4, wherein the duplex comprises an antisense oligonucleotide of miR-106b, the antisense oligonucleotide comprising the sequence AUCUGCACUGUCAGCACUUUA (SEQ ID NO: 6).

6. An antagonist of miR-106b expression, level, or activity.

7. The antagonist of claim 6, wherein the antagonist is an antisense oligonucleotide that is fully or partially complementary to at least a portion of miR-106b.

8. The antagonist of claim 7, wherein the miR-106b is human miR-106b-5p comprising the sequence UAAAGUGCUGACAGUGCAGAU (SEQ ID NO: 1).

9. The antagonist of claim 7, wherein the miR-106b is human miR-106b-3p comprising the sequence CCGCACUGUGGGUACUUGCUGC (SEQ ID NO: 2).

10. The antagonist of any one of claims 6-9, comprising DNA.

11. The antagonist of any one of claims 6-9, comprising RNA.

12. The antagonist of any one of claims 6-11, wherein the antagonist is an antagomir miR-106b.

13. The antagonist of any one of claims 7-12, wherein the antisense oligonucleotide comprises one or more nucleotide analogs.

14. The antagonist of claim 13, wherein the one or more nucleotide analogs comprises a locked nucleic acid (LNA).

15. The antagonist of any one of claims 7-14, wherein the antisense oligonucleotide is capable of forming a duplex with a mature miR-106b molecule, the duplex having a melting temperature (Tₘ) of at least about 60° C.

16. The antagonist of any one of claims 7-14, wherein the antisense oligonucleotide is capable of forming a duplex with another single stranded RNA molecule.

17. The antagonist of claim 16, wherein the other single stranded RNA molecule is a miR-93 RNA molecule.

18. The antagonist of claim 16, or 17, wherein the duplex of the antisense oligonucleotide and the other single stranded RNA molecule has a Tₘ of less than about 65° C., less than about 60° C., less than about 55° C., less than about 50° C., less than about 45° C., less than about 40° C., less than about 37° C., less than about 35° C., less than about 30° C., or less than about 25° C.

19. The antagonist of any one of claims 6-18, wherein
a. the antagonist is an antisense oligonucleotide that is fully or partially complementary to at least a portion of miR-106b;
b. the antisense oligonucleotide is capable of forming a duplex with a mature miR-106b molecule;
c. the antisense oligonucleotide is capable of forming a duplex with another single stranded RNA molecule; and
d. the Tₘ of a duplex formed by the antisense oligonucleotide with a mature miR-106b molecule is greater than the Tₘ of a duplex formed by the antisense oligonucleotide with the other single stranded RNA molecule.

20. The antagonist of claim 19, wherein the other single stranded RNA molecule is an miR-93 RNA molecule.

21. The antagonist of claim 20, wherein the miR-93 RNA molecule comprises the sequence AAAGUGCUGUUCGUGCAGGUAG (SEQ ID NO: 3) or the sequence
(SEQ ID NO: 4)
CAAAGUGCUGUUCGUGCAGGUAG.

22. The antagonist of any one of claims 6-21, wherein the antagonist is encoded by an isolated nucleic acid, or vector comprising the isolated nucleic acid.

23. The antagonist of claim 22, wherein said vector is an expression vector selected from an miRNA expression vector or AAV expression vector.

24. The antagonist of claim 23, wherein said expression vector is an miRNA expression vector.

25. The antagonist of claim 22, 23, or 24, wherein said isolated nucleic acid is operably-linked to a cell-specific promoter.

26. The antagonist of any one of claims 6-25 or the nucleic acid duplex of any one of claims 1-5, wherein the antagonist or nucleic acid duplex is encapsulated within a lipid vehicle.

27. A pharmaceutical composition comprising (a) an effective amount of the nucleic acid duplex of any one of claim 1-5 or 26 or the antagonist of any one of claims 6-26; and (b) a pharmaceutically acceptable carrier.

28. The pharmaceutical composition of claim 27, further comprising an additional therapeutic agent.

29. The pharmaceutical composition of claim 28, wherein said additional therapeutic agent comprises an anti-ischemia agent.

30. The pharmaceutical composition of any one of claims 27-29, wherein the effective amount is effective to decrease expression of at least one cell cycle pathway gene in an endothelial or muscle cell of a subject who is administered the pharmaceutical composition.

31. The pharmaceutical composition of claim 30, wherein said cell cycle pathway genes are selected from the group consisting of E2F-1 and p53.

32. The pharmaceutical composition of claim 30 or 31, wherein said expression is in skeletal muscle cells.

33. The pharmaceutical composition of any one of claims 27-32, wherein the effective amount is effective to enhance perfusion recovery in a subject who is administered the pharmaceutical composition.

34. The pharmaceutical composition of any one of claims 27-33, wherein the effective amount is effective to enhance angiogenic response to ischemia in a subject who is administered the pharmaceutical composition.

35. The pharmaceutical composition of any one of claims 27-34, wherein the effective amount is effective to stimulate cell proliferation.

36. The pharmaceutical composition of claim 35, wherein the cell proliferation comprises proliferation of endothelial cells or muscle cells.

37. The pharmaceutical composition of any one of claims 27-36, wherein the effective amount is effective to increase capillary density in a subject who is administered the pharmaceutical composition.

38. The pharmaceutical composition of any one of claims 27-37, wherein the effective amount is effective to inhibit apoptosis of one or more cells in a subject who is administered the pharmaceutical composition.

39. The pharmaceutical composition of claim 38, wherein said apoptosis is hypoxia-induced apoptosis.

40. The pharmaceutical composition of any one of claims 27-39, formulated for administration by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

41. The pharmaceutical composition of claim 40, wherein said administration is intramuscular.

42. A method of treating or preventing a disease, disorder, injury, or condition associated with ischemia, said method comprising administering to a subject in need thereof the pharmaceutical composition of any one of claims 27-41.

43. The method of any one the preceding claims, further comprising administering to the subject an additional therapeutic agent.

44. The method of claim 43, wherein said additional therapeutic agent comprises an anti-ischemia agent.

45. The method of any one of claims 42-44, wherein the effective amount is effective to decrease expression of, or attenuate ischemia-induced upregulation of, at least one cell cycle pathway gene in an endothelial or muscle cell of the subject.

46. The method of claim 45, wherein said cell cycle pathway genes are selected from the group consisting of E2F-1 and p53.

47. The method of any one of claims 42-46, wherein said expression is in skeletal muscle cells.

48. The method of any one of claims 42-47, wherein the effective amount is effective to enhance perfusion recovery in the subject.

49. The method of any one of claims 42-48, wherein the effective amount is effective to enhance angiogenic response to ischemia in the subject.

50. The method of any one of claims 42-49, wherein the effective amount is effective to stimulate cell proliferation.

51. The method of claim 50, wherein the cell proliferation comprises proliferation of endothelial cells or muscle cells.

52. The method of any one of claims 42-51, wherein the effective amount is effective to increase capillary density in the subject.

53. The method of any one of claims 42-52, wherein the effective amount is effective to inhibit apoptosis of one or more cells in the subject.

54. The method of claim 53, wherein said apoptosis is hypoxia-induced apoptosis.

55. The method of any one of claims 42-54, wherein said administration is by a route selected from the group consisting of oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, rectal, intrasternal injection, kidney dialytic infusion, and parenteral.

56. The method of claim 55, wherein said administration is intramuscular.

57. The method of any one of claims 42-55, wherein said subject is a human.

58. The method of any one of the claims 42-57, wherein said ischemia is selected from the group consisting of vascular ischemia, muscular ischemia, peripheral arterial disease, ischemia reperfusion injury, ischemia associated with trauma, and brain ischemia.

59. The method of claim 58, wherein the ischemia is peripheral arterial disease.
